(19) United States
(12) Patent Application Publication
(10) Pub. No.: US 2007/0073267 A1
(43) Pub. Date: Mar. 29, 2007

(54) Low-loss multi-lumen injection apparatus

(75) Inventor: David F. Muller, Boston, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: MILE CREEK CAPITAL, LLC, Salem, NH (US)

(21) Appl. No.: 11/337,788

(22) Filed: Jan. 23, 2006

Related U.S. Application Data
(63) Continuation-in-part of application No. 11/236,329, filed on Sep. 27, 2005.

(51) Int. Cl. A61M 31/00

(52) U.S. Cl. 604/506; 604/91; 604/191

(57) ABSTRACT

Injection devices, systems and methods are disclosed for injecting two or more medicaments to a patient at a single injection site while preferably minimizing any mixing of the medicaments prior to delivery to the patient. The invention can also be used to sequentially deliver the medicaments to the patient in a repetitive manner. For example, the injection apparatus can sequentially provide a first medicament and then a second medicament to the patient during a first injection procedure. During a second injection procedure, the injection apparatus can again sequentially provide the first medicament and the second medicament to the patient either at the injection site of the first injection procedure or at a different injection site. Multi-lumen manifolds are disclosed for coupling to conventional drug ampoules, to permit the user to sequentially delivery different medicaments via a single skin penetration and to reduce losses associated with usage. Systems including multiple drug reservoirs and filling adaptors are also disclosed.

### REFERENCE TO RELATED APPLICATION

This application is a continuation-in-part of U.S. Pat. Application Ser. No. 11/236,329, filed Sep. 27, 2005.

### FIELD OF THE INVENTION

The technical field of the invention concerns medicament injection methods and apparatus, and more particularly to injection techniques and devices for injecting two or more medicaments to a patient at a single injection site (or closely spaced sites).

### BACKGROUND OF THE INVENTION

Various devices have been developed for the subcutaneous delivery of medications or other treatment agents to a patient. For example, conventional injection devices include a syringe and a hollow needle. The needle is inserted under the skin of the patient and a syringe plunger delivers a medicament, typically a liquid medicament, contained within the syringe through the needle and into the skin.

There are various situations where it is desirable to deliver two medicaments. For example, injection devices are used to deliver wrinkle-reducing agents, such as botulism toxin (e.g., BOTOX) or collagen, to facial areas of a patient. Subcutaneous injection of such cosmetic agents, however, typically is painful. To minimize the pain associated with such injections, it may be advantageous to administer a local anesthetic at the target site prior to injection of the therapeutic or cosmetic agent.

In such applications, two separate injection devices would be needed: a first injection device containing an anesthetic compound and a second injection device containing the cosmetic agent. The needle of the first injection device must penetrate the target skin region so that the anesthetic agent can delivered to the patient, and then is withdrawn. Next, the needle of the second injection device penetrates the same site so that the cosmetic agent can be delivered.

This procedure would need to be repeated numerous times during a facial cosmetic treatment. While the use of the anesthetic can reduce pain during treatment, each injection site of the patient usually requires two separate needle insertions or needle “sticks”. Multiple needle insertions can traumatize the injection site and cause the patient's skin to become discolored (e.g., black-and-blue) at the site.

Registration of the first and second injections can also be difficult unless the target sites are marked in advance (e.g., by drawing dots on the patient's face with a marker). Moreover, the overall time to perform the treatment procedure is lengthened by the need for separate and sequential delivery of the different medicaments.

Simultaneous deliver of two medicaments may be desirable but is usually not practical. Even if two drugs could be mixed without side effects, the nature of the formulations (e.g., solvents, pH, and storage temperature constraints), would typically preclude direct co-administration.

Moreover, when two or more medicaments are applied together, it is often the case that one medicament is more expensive than the other and, hence, full utilization of the more expensive agent is desirable. In the course of treating a patient, it may be necessary to use several ampoules or doses of an expensive medicament.

Thus, there is a need for improved medicament delivery systems that allow independent subcutaneous delivery of multiple medicaments to a patient during a single injection while limiting mixing of the medicaments prior to delivery to the patient. In addition, systems that permit delivery of multiple medicaments with minimal or substantially no waste of medicament would be economically advantageous.

### SUMMARY OF THE INVENTION

Injection devices, systems, and methods are disclosed for injecting two or more medicaments to a patient at a single injection site. The injection apparatus is preferably adapted to minimize mixing of the medicaments prior to delivery to the patient.

The injection apparatus can be used to sequentially deliver two or more medicaments to the patient in a repetitive manner. For example, the injection apparatus can sequentially provide a first medicament and then a second medicament to the patient during a first injection procedure. The injection apparatus can then sequentially provide the first medicament and the second medicament to the patient during a second injection procedure either at the injection site of the first injection procedure or at a different injection site.

In one aspect of the invention, multi-lumen manifolds are disclosed for coupling to conventional drug syringes or other drug delivery sources, to permit the user to sequentially delivery different medicaments via a single skin penetration. A manifold according to the invention can included a manifold body, at least one injector adapted to penetrate skin, and at least two lumens disposed within the manifold body to provide fluidic coupling between separate medicament sources and a target tissue site. The injector can further include a hollow needle having a single or multiple lumens, e.g., concentric tubular needle elements or nested needle elements to deliver the different medicaments.

In another aspect of the invention, injection systems are disclosed that can include a hollow inner needle defining a first lumen fluidically couplable to a first medicament reservoir and a first fluid delivery actuator and a hollow outer needle having a distal end that at least partially surrounds the inner needle, the outer needle defining a second lumen fluidically couplable to a second medicament reservoir and a second fluid delivery actuator. The injection apparatus can be used to penetrate tissue and independently deliver at least two separate medicaments from the first and second medicament reservoirs to a patient.

In another aspect of the invention, filling adaptors are provided for filling a multi-lumen injection apparatus with a medicament. The filling adaptor can include a fitting for fluidically coupling the adaptor to container of a medicament and a body defining a fluid passageway from a coupled medicament container to a lumen of a multi-lumen injection apparatus. The filling adaptor can also include a lumen engagement portion configured to engage at least one lumen of a multi-lumen injection apparatus and block access of the medicament to the one lumen while the passageway is aligned with another lumen of the multi-lumen injection apparatus to allow passage of the medicament to the other lumen.

In another aspect of the invention, an injection kit is provided. The injection kit can include an injection apparatus for independently delivering a plurality of medicaments and one or more filling adaptors. The apparatus can include an inner needle coupled to a first medicament reservoir and an outer needle having a distal end that at least partially surrounds the inner needle, the outer needle coupled to a second medicament reservoir and a second fluid delivery actuator. The filling adaptor can include a first adaptor configured to fill the first medicament reservoir and a second adaptor configured to fill the second medicament reservoir.

In another aspect of the invention, a method delivering a plurality of medicaments is disclosed. A multi-lumen injection apparatus can be disposed adjacent to a target skin region, the injection apparatus having at least a first reservoir containing a first medicament and a second reservoir containing a second medicament. The apparatus can also include a hollow inner needle defining a first lumen fluidically couplable to a first medicament reservoir and a hollow outer needle having a distal end that at least partially surrounds the inner needle and defining a second lumen fluidically couplable to a second medicament reservoir. The skin region can then be penetrated with the injection apparatus and the first medicament and the second medicament applied.

In yet another aspect of the invention, injection manifolds are disclosed for delivering a plurality of medicaments, via a manifold body and at least one hypodermic needle adapted to penetrate skin; wherein the manifold body has at least two lumens or reservoirs that provide fluidic coupling between separate medicament sources and a target tissue site, each lumen having an associated actuator to dispense a medicament and at least one lumen and its associated actuator are configured to dispense medicament with substantially no residual volume. Hubless or “zero-loss” configurations are disclosed to reduce the residual losses of medicaments.

### BRIEF DESCRIPTION OF THE DRAWINGS

The invention will be more fully understood from the following detailed description taken in conjunction with the accompanying drawings, in which:

FIG. 1 is a cross-sectional schematic side view of a multi-lumen injection apparatus according to the invention.

FIG. 2 is a schematic illustration of a concentric dual plunger arrangement for an injection apparatus.

FIG. 3 is a cross-sectional top view of the apparatus of FIG. 1 along section line 3-3.

FIG. 4 is a schematic illustration of an alternative embodiment of the invention employing a split plunger arrangement.

FIG. 5 is a schematic view of a distal needle-coupling end cap for use within the embodiment of FIG. 4.

FIG. 6 is a schematic diagram of an automated injection system according to an embodiment of the invention.

FIG. 7 is a cross-sectional view of the injection manifold of FIG. 1 along section line 7-7. according to an embodiment of the invention.

FIG. 8 is a cross-sectional view of the injection manifold of FIG. 1 along section line 8-8. according to another embodiment of the invention.

FIG. 9 is a cross-sectional view of an injection manifold according to an embodiment of the invention.

FIG. 10 is a schematic side view of a filling adaptor for filling a multi-lumen injection apparatus with medicaments.

FIG. 11 is a cross-sectional view of an adaptor as shown schematically in FIG. 10 for filling an inner lumen of a multi-lumen injection apparatus.

FIG. 12 is a cross-sectional view of an adaptor as shown schematically in FIG. 10 for filling an outer lumen of a multi-lumen injection apparatus.

FIG. 13 is a cross-sectional view of an injection manifold having a single needle according to an embodiment of the invention.

FIG. 14 is a cross-sectional view of an injection manifold having a single needle according to another embodiment of the invention.

FIG. 15 is a schematic perspective view of another embodiment of an injection manifold according to the invention.

FIG. 15A is a cross-sectional view of the injection manifold of FIG. 15.

FIG. 15B is an enlarged cross-sectional view of the tip of injection manifold of FIG. 15.

### DETAILED DESCRIPTION

For a fuller appreciation of the invention, various terms used in this specification are defined. The term “medicament” as used herein is intended to encompass not only prescription and over-the-counter drugs, but also various other therapeutic and cosmetic agents, including, not limited to anesthetic agents, toxins, nerve relaxing agents, vitamins, collagen and other biocompatible fillers, sunscreens, pigmentation and/or skin bleaching agents, synthetic and natural agents, including small molecules and larger molecules, such as peptides, proteins, lymphokines, growth factors, hormones, antibodies, conjugates of antibodies with other agents, and other chemical or biological molecules that can provide desired effects when injected into biological tissue. The term “medicament” as used herein further encompasses water, saline, and lumen-cleaning or purging solutions.

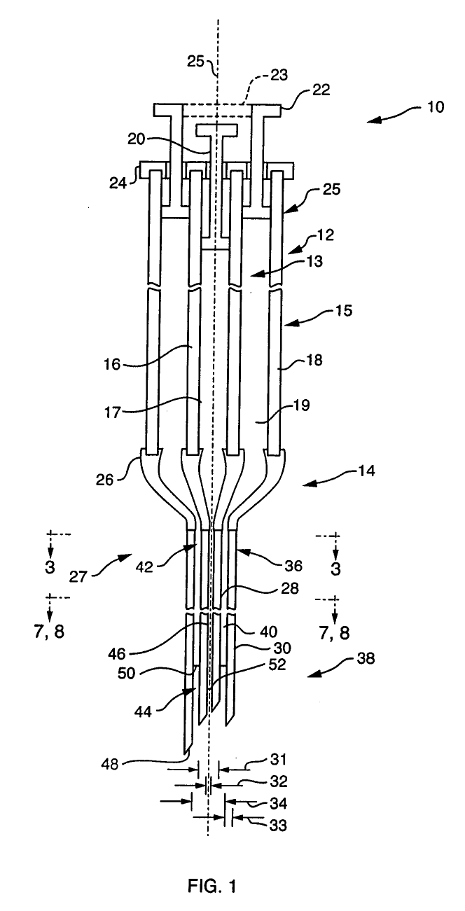

FIG. 1 illustrates an injection apparatus 10 in accordance with the present invention having a medicament delivery portion 12 and an injection manifold 14 in fluid communication with the medicament delivery portion 12. The medicament delivery portion 12 is configured to provide independent delivery of at least two separate medicaments to a patient via the injection manifold 14. The injection manifold 14 is configured to penetrate tissue of a patient and allow independent transmission of separate medicaments while limiting or preventing mixing of the medicaments prior to delivery to the patient.

The medicament delivery portion 12 can include a number of medicament reservoirs that contain and allow delivery of separate medicaments to a patient. As illustrated in FIG. 1, the medicament delivery portion 12 can include a first reservoir assembly 13 having a first reservoir 16 defining a first lumen 17 and a first actuator 20. The medicament delivery portion 12 can also include a second reservoir assembly 15 having a second reservoir 18 defining a second lumen 19 and a second actuator 22. In one embodiment, the first reservoir assembly 13 and the second reservoir assembly 15 can be configured as syringes where the first 16 and second 18 reservoirs are formed as substantially cylindrical syringe chambers and the first 20 and second 22 actuators are formed as syringe plungers.

Each of the first 16 and second 18 reservoirs can be configured to contain separate medicaments for delivery to the injection manifold 14. For example, the first reservoir 16 can be configured to contain a first medicament within the first lumen 17, such as an agent used in cosmetic surgery (e.g., BOTOX, collagen, RESTYLANE) and a second reservoir 18 can be configured to contain a second medicament within the second lumen 19, such as an anesthetic agent (e.g., Novocain, lidocaine).

Each of the first 20 and second 22 actuators can also be configured to provide independent delivery of the medicaments from each of the respective reservoirs 16, 18 to the injection manifold 14. For example, operation of the first actuator 20 (e.g., depression of the first syringe plunger) can deliver a portion of the medicament contained within the first reservoir 16 to the injection manifold 14 while operation of the second actuator 22 (e.g., depression of the second syringe plunger) can deliver a portion of the medicament contained in the second reservoir 18 to the injection manifold 14. With each actuator 20, 22 configured to operate independently from the other, the injection apparatus 10 can provide independent subcutaneous delivery of multiple medicaments to a patient during a single injection (i.e., at a single injection site).

The injection manifold 14 can couple to the medicament delivery portion 12 via a connector 26. For example, the connector 26 can be a threaded connector, a Luer locking element, or some other connecting element useful for providing a sealed fluidic coupling between the injection manifold 14 and the medicament delivery portion 12. In one embodiment, the connector 26 can align the lumens 17, 19 of the medicament reservoirs 16, 18 with corresponding hollow needles associated with the injection manifold 14 to provide fluid communication between the medicament delivery portion 12 and the injection manifold 14.

The injection manifold 14 can include a number of hollow needles configured to transmit medicaments from the medicament delivery portion 12 to a tissue while limiting mixing of the medicaments prior to delivery to the tissue. For example, in one embodiment, the injection manifold 14 can include an inner needle 28 in fluid communication with the first reservoir 16 and an outer needle 30 that at least partially surrounds the inner needle 28 and in fluid communication with the second reservoir 18. In another embodiment, the injection manifold 14 can include more than two needles. For example, the injection manifold 14 can include a two inner needles disposed within the outer needle 28, each inner needle in fluid communication with a corresponding (e.g., separate) reservoir assembly.

In one embodiment, the outer needle 30 includes a first end 36 that can couple to the second reservoir 18 via the connector 26, a second end 38 configured to insert within a tissue of a patient, and a lumen 40 extending between the first end 36 and the second end 38 and in fluid communication with the lumen 19 of the second reservoir 18. In one embodiment, the outer needle 30 can be smaller than a 28 gauge needle. For example, the outer needle can be a 30 gauge needle having an outer diameter 33 of approximately 0.31 mm and an inner (e.g., lumen) diameter 34 of approximately 0.19 mm.

In one embodiment, the inner needle 28 includes a first end 42 that can couple to the first reservoir 16 via the connector 26, a second end 44 configured to insert within a tissue of a patient, and a lumen 46 extending between the first end 42 and the second end 44 and in fluid communication with the lumen 17 of the first reservoir 16. In one embodiment, the inner needle 28 can be smaller than a 30 gauge needle. For example, the inner needle 28 can be a 36 gauge needle having an outer diameter of approximately 0.11 mm and an inner (e.g., lumen) diameter 32 of approximately 0.06 mm. In another example, the needle can be a 34.5 gauge needle having an outer diameter 31 of approximately 0.15 mm and an inner (e.g., lumen) diameter 32 of approximately 0.08 mm.

The relatively small sizes of the lumens 46, 40 of the needles 28, 30, combined with the positioning of the needles 28, 30 relative to each other (e.g., such as when the outer needle 30 surrounds a portion of the inner needle 28) can minimize an effect of capillary action within the lumens 46, 40 of the needles 28, 30. As such, the gauge size and relative positioning of the needles 28, 30 can minimize or prevent mixing or the exchange of medicaments between the needles 28, 30.

For example, regarding the capillary properties of a lumen or tube, generally the narrower the tube the further a liquid can be drawn within the tube. Referring to FIG. 1, assume a 36 gauge inner needle 28 inserts within a lumen 40 of a 30 gauge outer needle 30. Because the lumen 40 of the outer needle 30 can be relatively larger than the lumen 46 of the inner needle 28 (i.e., the lumen 46 of the inner needle 28 is narrower than the lumen 40 of the outer needle 30), the lumen 40 can draw a medicament to a lower (i.e., more proximal) level 50 within the injection manifold 14 relative to a higher (i.e., more distal) level 52 of the medicament drawn by the lumen 46. Such positioning of the medicaments within the lumens 46, 40 can minimize or prevent mixing of the medicaments carried by the needles 28, 30.

In one embodiment, one of the needles 28, 30 of the injection manifold 14 can be configured with a cutting surface to allow the injection manifold 14 to penetrate the tissue of a patient. For example, the second end (e.g., distal tip) 38 of the outer needle 30 can be configured with a beveled edge 48 that can cut into tissue and allow insertion of the outer needle 30 and the inner needle 28 into the tissue of a patient. In another embodiment, one of the needles 28, 30 of the injection manifold 14 can be configured with a non-cutting surface. For example, the second end (e.g., distal tip) 44 of the inner needle 30 can be configured as having a blunt (e.g., non-cutting) surface.

In one embodiment, the second end 44 of the inner needle 28 can be recessed (e.g., shorter) relative to the distal tip 38 of the outer needle 30 to minimize contact between the inner needle 28 and the tissue of a patient as the outer needle 30 inserts within the tissue of the patient during an injection procedure. As such, the recess limits axial loading of the inner needle 28 by the tissue and thereby minimizes the possibility of the inner needle 28 bending or fracturing during an injection procedure.

During operation, the injection apparatus 10 can deliver medicaments carried by the injection apparatus 10 to a patient. For example, the injection apparatus 10 can penetrate a skin region of a patient, such as at an injection site. In one embodiment, the distal tip 38 of the outer needle 30 can pierce the tissue of the patient at the injection site such that the distal tip 38 of the outer needle 30 and the distal tip 44 of the inner needle 28 insert within the tissue. The injection apparatus 10 can then deliver a first medicament to the patient at the injection site. For example, the second actuator 22 associated with the second reservoir 18 can be actuated to deliver a portion of the medicament contained in the second reservoir 18, such as an anesthetic agent, through the lumen 40 of the outer needle 30 and into the tissue.

Following injection of the first medicament, the injection apparatus 10 can then deliver a second medicament to the injection site either at substantially the same depth as the first medicament or at a different depth than the first medicament. In one embodiment, the position of the injection apparatus can be maintained relative to the injection site, thereby maintaining the relative position of the inner needle within the tissue. In another embodiment, the injection apparatus 10 can advance into the tissue such that the inner needle 28 positions at a different depth than the outer needle 30. The first actuator 20 associated with the first reservoir 16 can then be actuated, independently from the second actuator 22, to deliver a portion of the medicament contained in the first reservoir 16, such as a cosmetic agent, through the lumen 46 of the inner needle 28 and into the tissue.

Also during operation, the medicament delivery process described above can be repeated to deliver additional doses of the medicaments to the patient. In one embodiment, each medicament from the first 16 and second 18 reservoirs can be repeatedly delivered to the patient at the same injection site. For example, after the apparatus 10 has delivered doses of the first and second medicaments to the tissue, the apparatus 10 can deliver a second dose of the first and second medicaments to the tissue. In another embodiment, after delivery of the first and second medicaments to the patient at a first injection site, the injection manifold 14 can be removed from the first injection site and inserted within a second (e.g., different) injection site of the patient. Alternating delivery of the first and second medicaments from the first 16 and second 18 reservoirs can then be repeated for the second, and subsequent, injection sites of the patient.

As indicated above, the injection apparatus 10 can allow independent subcutaneous delivery of multiple medicaments to a patient during a single injection while limiting mixing of the medicaments prior to delivery to a patient. The injection apparatus 10 can be configured in a variety of ways to allow such medicament delivery to the patient.

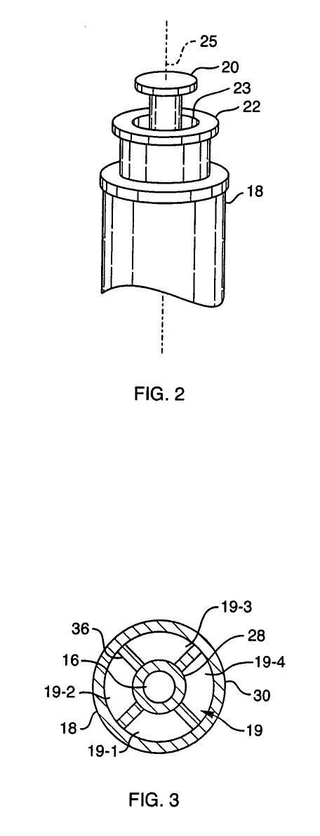

In one example, the reservoir assemblies 13, 15 can be positioned relative to each other in various configurations. In one embodiment as illustrated in FIG. 1, the first reservoir assembly 13 is disposed within the second reservoir assembly 15. For example, the first reservoir assembly 13 can be positioned concentrically relative to the second reservoir assembly 15 such that the first actuator 20 and the second actuator 22 align along a common longitudinal axis 25, as also illustrated in FIG. 2. With such a configuration, the second actuator 22 can define an opening 23 through which the second first actuator 20 can extend, thereby allowing independent actuation of the first 20 and second 22 actuators.

Returning to FIG. 1, with the first reservoir assembly 13 is disposed within the second reservoir assembly 15, the medicament delivery portion 12 can include structures that couple the fluid reservoirs 16, 18 to each other. For example, the medicament delivery portion 12 can include an end cap 24, such as disposed at a first or proximal end 25 of the medicament delivery portion 12 and one or more struts 33, such as disposed at a second or distal end 27 of the medicament delivery portion 12.

The end cap 24 can couple the first reservoir 16 and the second reservoir 18 to provide a degree of rigidity or stability to the apparatus 10. The end cap 24 can also maintain a spaced-apart relationship between the first reservoir 16 and the second reservoir 18 at the proximal end 25 of the medicament delivery portion 12. For example, the end cap 24 orients the first reservoir 16 relative to the second reservoir 18 to define a toroid-shaped lumen 19 between the walls of the reservoirs 16, 18 for containment of a medicament within the second reservoir 18.

As shown in FIGS. 1 and 3, the struts 36 are disposed between the first reservoir 16 and the second reservoir 18 and can couple the first reservoir 16 and the second reservoir 18 to provide a degree of rigidity or stability to the apparatus 10. The struts 36 can also maintain a spaced-apart relationship between the first reservoir 16 and the second reservoir 18 at the distal end 25 of the medicament delivery portion 12. For example, the struts 36 can divide the lumen 19 formed between the walls of first reservoir 16 and the second reservoir 18 into subsections, such as a first lumen 19-1, a second lumen 19-2, a third lumen 19-3, and a fourth lumen 19-4 to maintain a fluid pathway between the second reservoir 18 and the injection manifold 14.

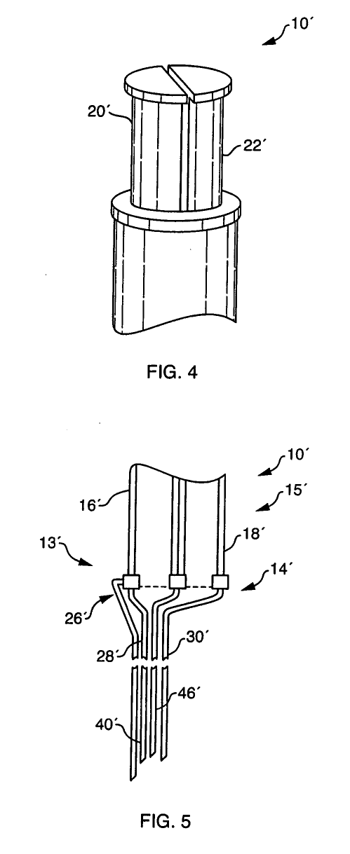

While the reservoir assemblies 13, 15 can be positioned within one another, as described above, the reservoir assemblies 13, 15 can also be positioned in a side-by-side or adjacent relationship. For example, FIGS. 4 and 5 illustrate an embodiment of the injection apparatus 10′configured with a first reservoir assembly 13′, having a first reservoir 16′ and a first actuator 20′, positioned adjacent to a second reservoir assembly 15′ having a second reservoir 18′ and a second actuator 22′. As shown in FIG. 4, the actuators 20′, 22′ can be configured as independently actuatable in a “split plunger” arrangement to allow independent delivery of medicaments to an injection site.

With the reservoir assemblies 13′, 15′ positioned in a side-by-side or adjacent relationship, the injection manifold can be configured to attach to the adjacent reservoir assemblies 13′, 15′ to provide fluid communication between the associated reservoirs 16′, 18′and the needles of the injection manifold. For example, the injection apparatus 10′, as shown in FIG. 5, can include an injection manifold 14′ coupled to the first 16′ and second 18′ reservoirs and having an inner needle 28′ and an outer needle 30′. While the needles 28′, 30′ are illustrated as being substantially aligned (e.g., aligned along a longitudinal axis) with the first reservoir 16′, the needles 28′, 30′ can be offset relative to the second reservoir 18′ (e.g., offset relative to a longitudinal axis of the second reservoir 18′). Such an offset provides fluid communication between the adjacently positioned reservoirs 16′, 18′ and the respective needles 28′, 30′.

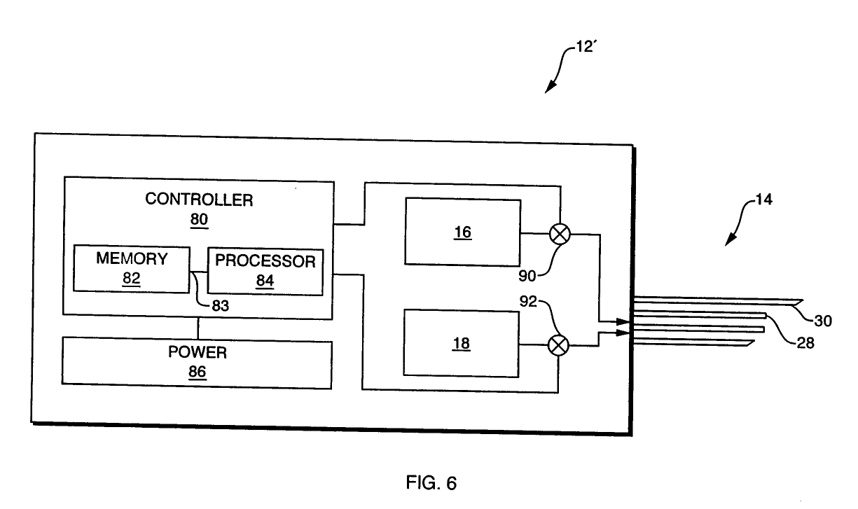

The actuators 20, 22 of the injection assembly 10 can be manually operated to deliver medicaments from the reservoirs 16, 18 to the injection manifold 14. However in one embodiment, operation of the actuators can be automated. FIG. 6 illustrates an embodiment of a medicament delivery portion 12′ of an injection assembly 10 having automated actuators.

The medicament delivery portion 12′ can include a controller 80 in electrical communication with actuators 90, 92 and electrically couples to a power supply 86, such as either an internal power supply (e.g., a battery) or an external power supply. The first actuator 90, such as a valve, can position in fluid communication with the first reservoir 16 and with the inner needle 28. The second actuator 92, such as a valve, can position in fluid communication with the second reservoir 18 and the outer needle 30.

The controller 80 can include a memory 82 and a processor 84. The memory 82 can be any type of computer readable medium such as electronic semiconductor memory (e.g., Random Access Memory or Read Only Memory), programmable memory (e.g., EEPROM), or another storage or enclosable medium such magnetic or optical disk storage. The processor 84 can be any type of central processing unit, microprocessor, programmable gate array (PGA) or other circuitry that are capable of executing, interpreting, operating, being configured with, or otherwise performing sets of logic instructions such as computer program code. A data bus 83 or other circuitry can interconnect the memory 82 and the processor 84.

The controller 80, in one embodiment, is configured to control delivery of a proscribed dosage (e.g., volume) of the medicaments contained within the reservoirs 16, 18 to an injection site. For example, based upon instructions stored in the memory 82 and executed by the processor 84, at a first time, the controller 80 can send a first signal to the first actuator 90 and to the second actuator 92. The first signal can cause the first actuator 90 to prevent the flow of medicament from the first reservoir 16 to the inner needle 28 and can cause the second actuator 92 to allow a flow of medicament from the second reservoir 18 to the outer needle 30. After delivery of a proscribed dosage of the medicament from the first reservoir 16, the controller 50 can then send a second signal to the first 90 and second 92 actuators that can cause the first actuator 90 to allow a flow of medicament from the first reservoir 16 to the inner needle 28 and can cause the second actuator 92 to prevent a flow of medicament from the second reservoir 18 to the outer needle 30. Such automated delivery can provide sequential delivery of medicaments to a single injection site and can allow delivery of substantially precise dosages of the medicaments to the site.

As indicated above, the injection manifold 14 can deliver multiple medicaments to a patient during a single injection while limiting or preventing mixing of the medicaments prior to delivery to the patient. The injection manifold 14 can be configured in a variety of ways to allow such medicament delivery to the patient.

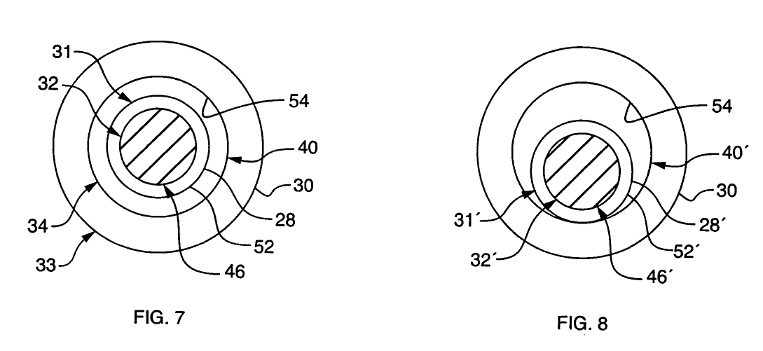

In one embodiment, the needles 28, 30 are oriented substantially coaxially relative to each other. For example, FIG. 7 illustrates an embodiment of a 30 gauge inner needle 28 coaxially disposed within a 36 gauge outer needle 30. The 30 gauge inner needle 28 defines a lumen 46 having cross-sectional area of approximately 2800μm². The annular cross-sectional area of a lumen 40 formed between an outer wall 52 of the inner needle 28 and an inner wall 54 of the outer needle 30 is approximately 18,800μm². The relatively large lumen 40 formed between the outer needle 28 and the inner needle 28 reduces the capillary properties of the lumen 40 and minimizes mixing of the medicaments carried by the needles 28, 30.

In another embodiment, the needles 28, 30 are oriented in a substantially eccentric manner relative to each other. FIG. 8 illustrates an embodiment of a 34.5 gauge inner needle 28′eccentrically disposed within the 36 gauge outer needle 30 (e.g., a longitudinal axis of the inner needle 28′ is offset from a longitudinal axis of the outer needle 30). The 30 gauge inner needle 28′ has a lumen 50′ having a cross-sectional area of approximately 5000μm². The annular cross-sectional area of a lumen 58′ formed between an outer wall 52′ of the inner needle 28′ and an inner wall 54′ of the outer needle 30′ is approximately 10,700μm². The relatively large lumen 58′ formed between the outer needle 28′ and the inner needle 28′ reduces the capillary properties of the lumen 58′, compared to the relatively small lumen 50′ of the inner needle 28′, and minimizes mixing of the medicaments carried by the needles 28′, 30′.

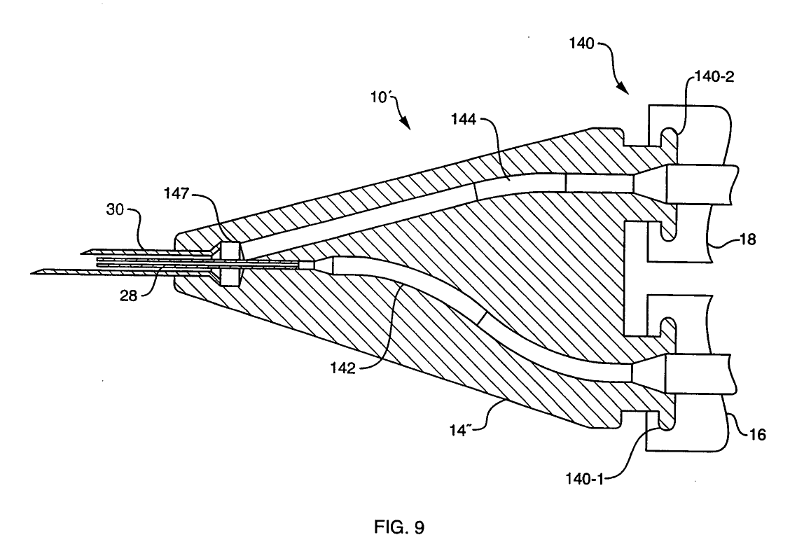

In one embodiment, the injection manifold 14 can include connection elements that allow one or more reservoir assemblies to attach to the injection manifold 14. For example, FIG. 9 illustrates an embodiment of the injection manifold 14″ having hubs 140 configured to allow attachment of medicament reservoirs to the injection manifold 14″. In one embodiment, the injection manifold 14″ includes a first hub 140-1 and a second hub 140-2 for attachment of a first medicament reservoir 16 and a second medicament reservoir 18, respectfully.

The injection manifold 14″ can include fluid pathways formed between the hubs 40 and corresponding needles. In one embodiment, the injection manifold 14″ can include a first fluid pathway 142 between the first hub 140-1 and the inner needle 28 for transmission of a first medicament from the first reservoir 16 to the inner needle 28 and can also include a second fluid pathway 144 between the second hub 140-2 and the outer needle 30 for transmission of a second medicament from the second reservoir 18 to the outer needle 30. In one embodiment, the injection manifold 14″ can include a distributor 147 positioned between the second fluid pathway 144 and the outer needle 30. The distributor 147 can be configured to provide a substantially uniform distribution of the second medicament from the second fluid pathway 144 to the outer needle 30.

The injection apparatus 10 can be provided prior to use (e.g., such as by a manufacturer) with each of the medicament reservoirs filled with a separate (e.g., distinct) medicament. However, in certain cases, the injection apparatus 10 can be provided with substantially empty medicament reservoirs that require filling prior to use of the injection apparatus 10. As indicated above, however, the injection assembly 10 can include two or more needles, each in fluid communication with a medicament reservoir. In one embodiment, a filling adaptor can be provided to allow the medicament reservoirs of the injection apparatus 10 to be selectively filled prior to use using the needles of the apparatus 10.

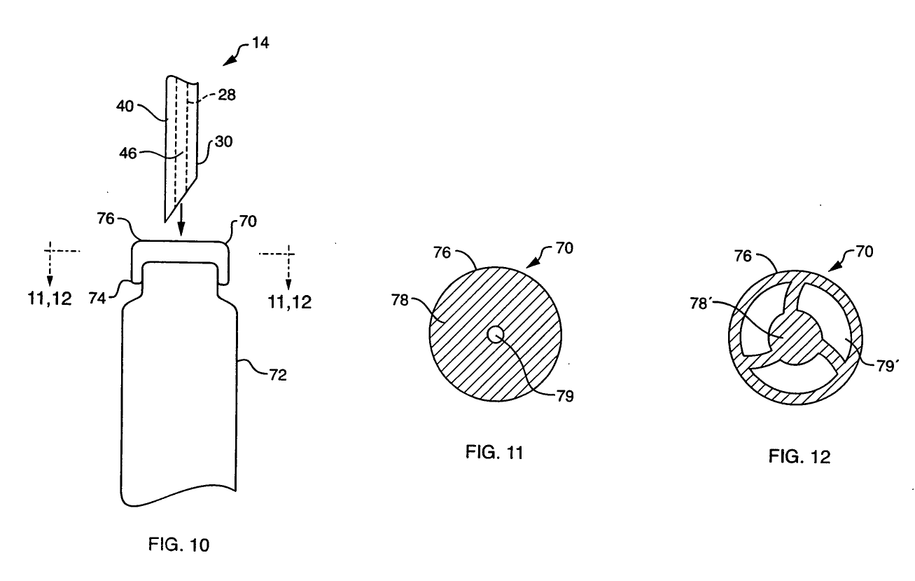

FIG. 10 illustrates a filling adaptor 70 that can provide the medicament reservoirs (e.g., lumens 17, 19 of the reservoirs 16, 18) with access to a medicament, such as contained within a container 72. In one embodiment, the adaptor 70 can include a fitting portion 74 and a body portion 76. The fitting portion 74 can be configured to couple the adaptor 70 to the container 72 and the body portion 76 can be configured to provide selective access to the medicament within the container. As will be described with respect to FIGS. 11 and 12, the body portion 76 can define a fluid passageway that provides a first lumen of the injection assembly 10 access to the medicament and can also include a lumen engagement portion, such as formed as an elastomeric pad, that can block a second lumen of the injection assembly 10 access to the medicament.

FIG. 11 illustrates an embodiment of the adaptor 70 configured to provide medicament access to an inner needle 28, and an associated reservoir 16 of the injection assembly 10. The body portion 76 can define an opening 79 oriented substantially at a central location of the adaptor 70 and configured to align with the lumen 46 of the inner needle 28. The body portion 76 can also include an engagement portion 78 oriented substantially about a circumference of the adaptor 70 and configured to align with or engage the lumen 40 between the inner needle 28 and the outer needle 30.

During operation, as the injection manifold 14 inserts within the adaptor, the inner needle 28 can insert within the opening 79 and the outer needle 30 can engage the engagement portion 78. As a result, the lumen 46 of the inner needle 28 can access the medicament contained in the container 72, thereby allowing the associated medicament reservoir 16 to be filled with the medicament. Additionally, the material forming the engagement portion 78 can fill or substantially encompass a cross-sectional area of the lumen 40 between the inner needle 28 and the outer needle 30 thereby limiting or preventing the lumen's 40 access to the medicament contained in the container 72 and preventing the associated medicament reservoir 18 from being filled with the medicament.

FIG. 12 illustrates an embodiment of the adaptor 70 configured to provide medicament access to an outer needle 30, and an associated reservoir 18 of the injection assembly 10. The body portion 76 can define one or more openings 79′ positioned substantially about a circumference of the adaptor 70 and configured to align with the lumen 40 between the inner needle 28 and the outer needle 30. The body portion 76 can also include an engagement portion 78′ oriented substantially at a central location of the adaptor 70 and configured to align with the lumen 46 of the inner needle 28.

During operation, as the injection manifold 14 inserts within the adaptor, the outer needle 30 can insert within the opening 79′ and the inner needle 28 can engage the engagement portion 78′. As a result, the lumen 40 between the inner needle 28 and the outer needle 30 can access the medicament contained in the container 72, thereby allowing the associated medicament reservoir 18 to be filled with the medicament. Additionally, the material forming the engagement portion 78′ can fill or substantially encompass a cross-sectional area the lumen 46 of the inner needle 28 thereby limiting or preventing the lumen's 46 access to the medicament contained in the container 72 and preventing the associated medicament reservoir 16 from being filled with the medicament.

As indicated above, the injection manifold 14 is configured to penetrate tissue of a patient and allow independent transmission of separate medicaments while limiting or preventing mixing of the medicaments prior to delivery to the patient. As described in FIGS. 7-9, the injection manifold can include a number of concentric or otherwise nested needles configured to transmit medicaments from the medicament delivery portion 12 to the patient. However, in other embodiments, the injection manifold 14 includes a single needle that allows independent transmission of separate medicaments while limiting or preventing mixing of the medicaments prior to delivery to the patient.

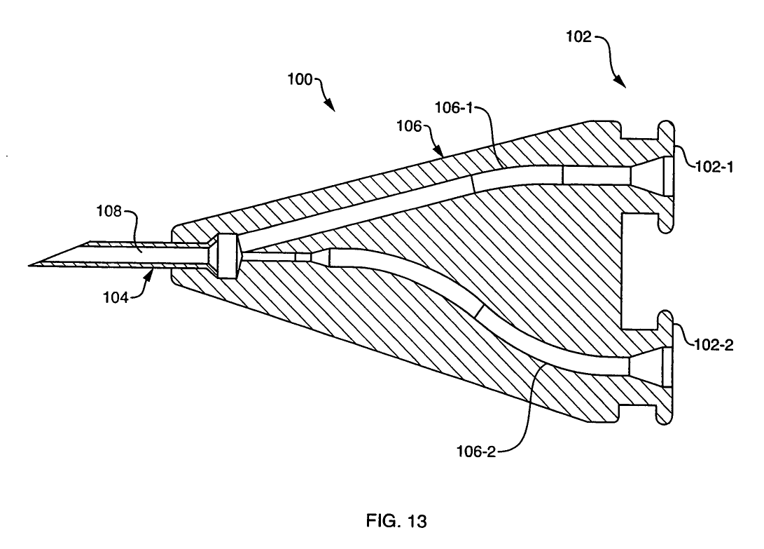

FIG. 13 illustrates an embodiment of an injection manifold 100 having ports 102, a needle 104, and fluid pathways 106 disposed between the needle 104 and the ports 102. The injection manifold 100 can include a first port 102-1 and a second port 102-2 that can be configured to couple to drug sources, e.g. drug-loaded syringes, containing distinct medicaments. The fluid pathways can include a first pathway 106-1 and a second fluid pathway 106-2 in fluid communication with the first port 102-1 and the second port 102-2 and in fluid communication with a lumen 108 defined by the needle 104. The needle lumen 108 can have a diameter that minimizes or prevents the medicaments sequentially delivered from the first port 102-1 and the second port 102-2 from mixing within the needle 104. For example, in one embodiment the needle 104 can be configured as a 30 gauge needle (e.g., having a diameter of approximately 0.19 mm).

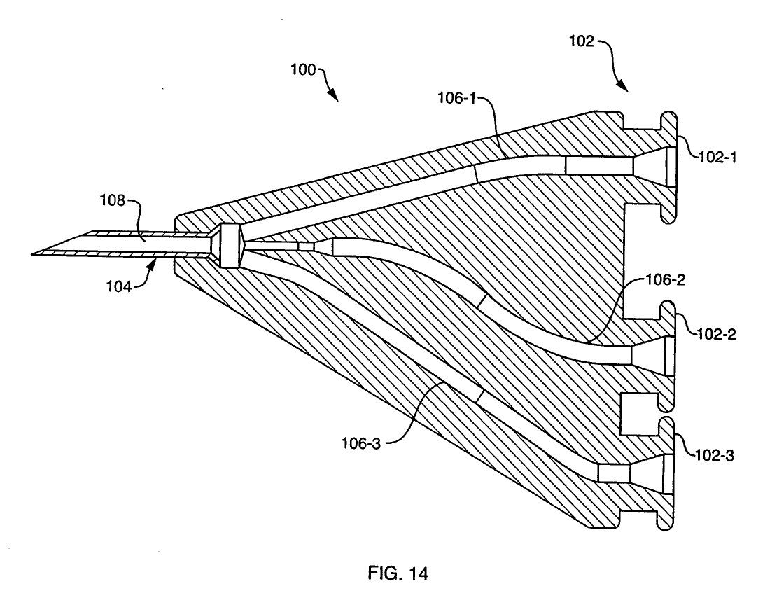

While the embodiment of the injection manifold 100 illustrated in FIG. 13 is shown as having two ports 102 and two fluid pathways 106, the injection manifold 100 can have more than two ports 102 and two fluid pathways 106. For example, FIG. 14 illustrates another embodiment of an injection manifold 100 having a first port 102-1, a second port 102-2, and a third port 102-3 configured to couple to a third reservoir. The injection manifold 100 can also have a third fluid pathway 106-3 disposed between the third port 106-3 and the lumen 108 of the needle 104.

In one embodiment, the third port 102-3 can couple to a reservoir containing a saline solution. In such an embodiment, the third fluid pathway 106-3 is configured to direct saline from the reservoir and through the lumen 104 of the needle 108 prior to delivery of a first medicament from the first port 102-1 to the needle 104 and prior to delivery of a second medicament from the second port 102-2 to the needle 104. The saline can flush the lumen 108 of the needle prior to delivery of the medicament from either port 102-1, 102-2, thereby minimizing mixing of medicaments within the needle 104.

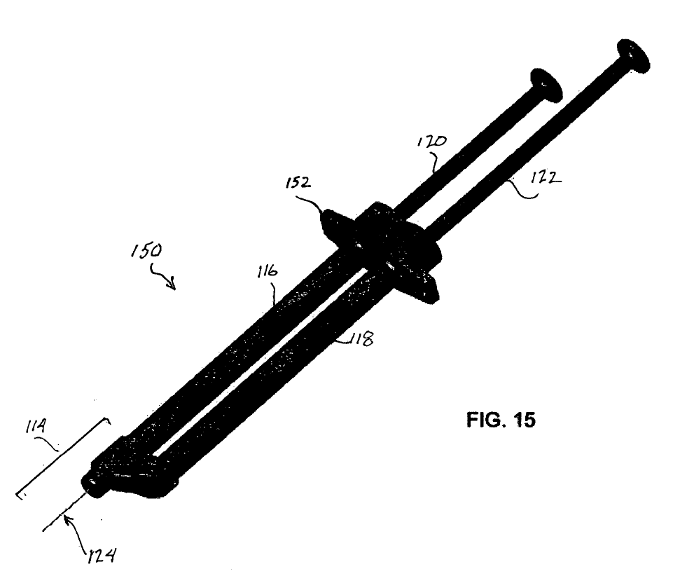

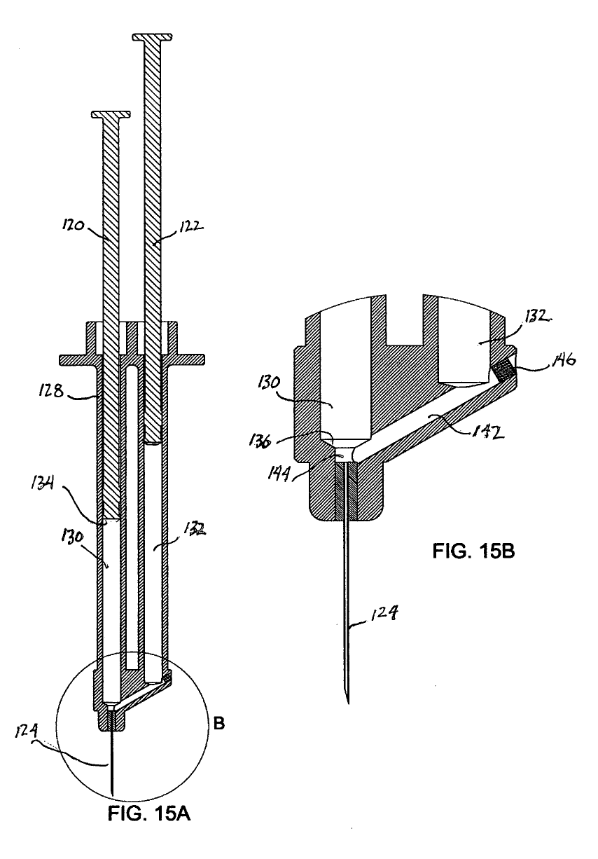

FIGS. 15, 15A and 15B illustrate yet another embodiment of the invention in which a low-loss injection manifold 150 is shown having manifold body 152 defining two medicament reservoirs 116 and 118, a distal injector 114 and two plunger-type actuators 120 and 122 for manual dispensing of medicaments. As shown in more detail in the cross-sectional views of FIGS. 15A and 15B, reservoir 116 includes an inner lumen 130, through which the distal end 134 of plunger 120 passes to dispense medicament from reservoir 116 via needle 124. The distal wall 136 of the lumen 130 is configured to receive the distal end 134 of plunger 120 in a mating relationship (e.g., matching frusco-conical shapes) such that there is substantially no residual volume or waste of medicament (e.g., less than 10 microliters, preferably less than 5 microliters, more preferably less than 1 microliter in certain applications). Essentially, the only residual medicament (following full depression of plunger 120) will be in antechamber 144 and the needle itself. Lumen 132, which permits delivery of a second separate medicament, can also be designed for substantially no residual medicament by appropriate sizing of passageway 142. Optionally, the injection manifold can further include an access port 146 for purging passageway 142, antechamber 144 and needle 124. Although the device of FIGS. 15, 15A and 15B is shown with two lumens, it should be clear three or more reservoirs can likewise be constructed for delivery of multiple medicaments.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated by reference in their entirety.

What is claimed is:

1. An injection manifold for delivering a plurality of medicaments, the apparatus comprising:
a manifold body;
at least one hypodermic needle adapted to penetrate skin; and
at least two lumens disposed within the manifold body to provide fluidic coupling between separate medicament sources and a target tissue site, each lumen having an associated actuator to dispense a medicament,
wherein at least one lumen and its associated actuator are configured to dispense medicament with substantially no residual volume.

2. The injection manifold of claim 1 wherein at least one lumen has an associated actuator that further comprises a plunger slidable disposed within the lumen for longitudinal movement to dispense medicament from a distal end of the lumen.

3. The injection manifold of claim 2 wherein the plunger and lumen are configured such that when a medicament is dispensed from the lumen and the plunger has reached the distal end of the lumen, the residual volume of medicament within the manifold is less than 10 microliters.

4. The injection manifold of claim 3 wherein the residual volume is less than 5 microliters.

5. The injection manifold of claim 3 wherein the residual volume is less than 1 microliters.

6. The injection manifold of claim 1 wherein the hypodermic needle is single lumen needle.

7. A method for delivering a plurality of medicaments, the method comprising:
disposing an injection apparatus adjacent to a target skin region;
penetrating the skin region with the injection apparatus;
applying a first medicament using the injection apparatus;
applying a second medicament using the injection apparatus; and
repeating the steps of applying the first medicament and applying the second medicament using the injection apparatus,
at least one of said medicaments being applied via a manifold that has substantially no residual volume.

8. A method for delivering a plurality of medicaments, the method comprising:
disposing a multi-lumen injection apparatus adjacent to a target skin region, the injection apparatus having a manifold body, at least one hypodermic needle adapted to penetrate skin; and at least two lumens disposed within the manifold body containing separate medicament sources, each lumen having an associated actuator to dispense a medicament, and wherein at least one lumen and its associated actuator are configured to dispense medicament with substantially no residual volume;
penetrating the skin region with the injection apparatus;
applying one medicament; and
applying other medicament.

9. The method of claim 8 wherein the method further comprises penetrating the skin to an initial depth and releasing one medicament and then penetrating to a different depth and release another medicament.

10. The method of claim 8 wherein the method further comprises sequentially applying at least two different medicaments from the first and second reservoirs repeatedly at a same location.

11. The method of claim 8 wherein the method further comprises repeating the application of the at least two different medicaments from the first and second reservoirs at a different location.

12. The method of claim 8 wherein one medicament is an anesthetic agent and the method further comprises initially applying the anesthetic agent from one of the reservoirs.
